(19) United States
(12) Patent Application Publication
(10) Pub. No.: US 2011/0060414 A1
(43) Pub. Date: Mar. 10, 2011

(54) ADHESIVE, SHOCK-ABSORBING INTERVERTEBRAL DISK PROSTHESIS

(76) Inventor: Frederic Fortin, Pessac (FR)

(21) Appl. No.: 11/817,680

(22) PCT Filed: Feb. 28, 2006
(86) PCT No.: PCT/FR06/00440

§ 371 Date: Sep. 1, 2007

(30) Foreign Application Priority Data
Mar. 2, 2005 (FR) 0502092

(51) Int. Cl. A61F 2/44

(52) U.S. Cl. 623/17.16

(57) ABSTRACT

The invention relates to an adhesive, shock-absorbing prosthesis (1) comprising two perfectly-integrated means, namely: a homogeneous block (11) which is made from a flexible, shock-absorbing material that is resistant to compression pressures, i.e. a polyurethane-type material that can withstand the repeated mechanical stresses of the intervertebral disk; and at least one adhesion means (12 or 13) which is used to provide suction such that the prosthesis (1) can be fixed in a flexible manner.

### FIELD OF THE INVENTION

The present invention relates to an intervertebral disk prosthesis to be inserted between vertebras. It has new means of adhesion that enables it to remain in place without risk of expulsion under the stresses of the human body.

### PRIOR ART

We will briefly point out the constitution of an intervertebral disk binding two vertebral bodies.

Since the works of Van Steen Brugghe in 1956, many patents cite various intervertebral disk implementations.

Currently, the concepts of disk prostheses can be classified according to three groups:

total intervertebral disk prostheses

pulposus nucleus prostheses

annulus fibrosis prostheses

Various prosthesis systems exist on the market. They are devices composed of different elements that are not easily implantable, because they impose heavy and traumatizing surgical acts. They are employed when the intervertebral disk to be treated is already degraded. The complex assembly of these multiple parts that constitute this type of system reduces the quality and the effectiveness of function over time, and moreover it is very difficult to ensure their installation, because these prostheses have a tendency to be expelled under the stresses of the human body.

There are several surgical methods using various of implants types to treat pathologies of the intervertebral disk when treatments by medications or kinesitherapy become insufficient. The most prevalent treatments are ablations of the slipped disks and vertebral fusions.

The formation of a slipped disk is provoked by a tearing of fibrosis tissue of the disk (the annulus) that allows a part of the nucleus composed of gelatinous cells to protrude. These degenerated cells aggregate and lodge, for the more severe hernias, between the nervous system and the vertebra by forming a ball of material that compresses the nervous root of the spinal marrow, causing intense pains, necessitating a rapid surgical intervention. After this treatment, the patient is generally relieved and can resume normal activity. Nevertheless a good number of these patients will soon have other pains. In fact the cells expelled from the intervertebral disk will not be replaced by new healthy cells that would reform themselves “in situ”. There is no spontaneous healing as for the skin and the bones. This is why there generally remains a lack of nucleus in the disk cavity, which is will be a source of instability of the vertical column. The damaged disk will generally collapse further, allowing escape of the other nucleus cells, again provoking pains. It is matter of in this case of a recurrence of the disk hernia. On the other hand to compensate for the insufficiency of the disk, the articular facets situated on the posterior arc of the vertebras will be overloaded and their articular surface risks degradation, causing other pains.

When the functionalities of the disk and the vertebral connections are too affected, the only solution is then to make a bone graft between the vertebras, restoring an intervertebral space sufficient to avoid any compression of the nervous system. This is vertebral fusion. The latter is carried out with implants such as cages, screws, and rods, which operate by blocking all movement of the two vertebras, to support the formation of a bone bridge. But this vertebral fusion has harmful consequences in the medium term on the distribution of the forces of the others elements of the vertical column.

In fact the healthy intervertebral disk situated at the top of that which has been replaced by bone will be extremely stressed. It will compensate for the absence of mobility of the first. Thus the risk of degeneration of the upper intervertebral disk is elevated, if the patient resumes an intense activity. This entails then another surgical operation for the upper disk, and so on . . . One understands the necessity of definitively dealing with the problem from the start of the first symptoms, to avoid the sufferings and the risk of a cascade of surgical interventions, the result of which is an outcome of a gradual blocking of the back which involves irreversible degradations of the physical capacities of the patient.

A first invention patented under the number EP0919209 describes a nucleus prosthesis composed of a dry hydrogel block that, once implanted, will absorb of water and inflate at least a millimeter in height. The surgeon must first make a sufficiently broad incision in the annulus to introduce the prosthesis and to make it swivel a quarter of turn in its implantation site so that largest side of the parallelepiped rectangle faces back to the incision of the annulus. It is in fact essential that the prosthesis does not re-emerge from the cavity that it occupies. The already damaged annulus, the tear of which one has aggravated, plays an essential role of fortification to maintain the implant in place in the cavity left by the nucleus. One understands perfectly that a fortification that has a broad opening that one cannot re-close cannot fulfill this function, which explains the numerous cases of prostheses expelled after their installation.

A second invention patented under the number WO/99/02108 describes and claims a prosthesis nucleus composed of a bag into which the surgeon injects a polymer in situ to fill the cavity of the nucleus.

A third invention patented under the number FR 0111905 describes and claims a two-part prosthesis nucleus.
a first part that compresses in an insertion tool, the end of which penetrates in the nucleus by a lesion of the annulus; freed from the tool, this first part expands to occupy a pattern cavity approximately double the diameter of the hole made by the annulus
a second part that then clicks into the first in order to prevent the first part from re-closing and to occupy vacant volume left by the first part. This invention also uses the annulus as fortification for its stability.

In these two types of invention the risk of expulsion although diminished is unfortunately not null.

The present invention, by the installation of new means, has for essential purpose to render almost null the risk of expulsion, while keeping good mechanical characteristics of the prosthesis.

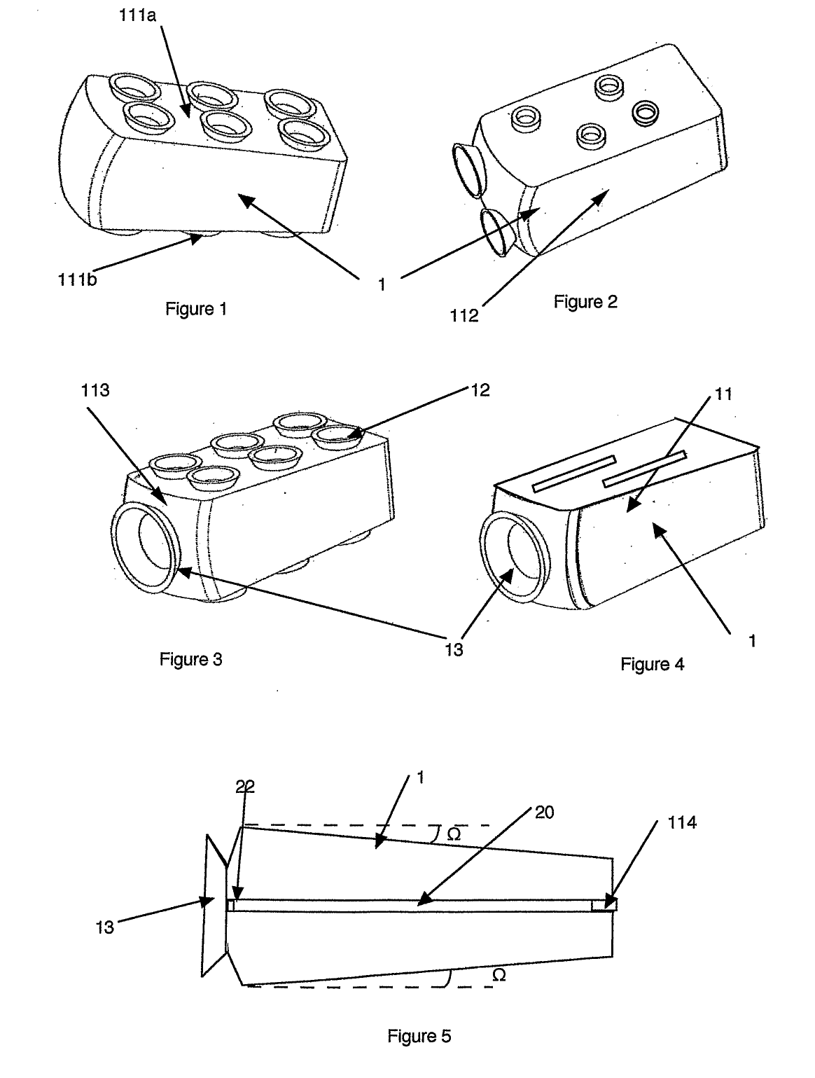

FIG. 1 of sheet 1/3 shows a perspective view of the prosthesis having a first implementation form of means of adhesion of the suction cup type.

FIG. 2 of sheet 1/3 shows a perspective view of the prosthesis having a second implementation form of means of adhesion of the suction cup type.

FIG. 3 of sheet 1/3 shows a perspective view of a prosthesis equipped with its adhesion devices formed from suction cups on the support faces and from at least one suction cup at the end that adheres at the bottom from the cavity.

FIG. 4 of sheet 1/3 shows a perspective view of a prosthesis equipped with its adhesion devices formed from least one suction cup situated at the end and that adheres at the bottom from the cavity.

FIG. 5 of sheet 1/3 shows a cross-section view of a shock-absorbing prosthesis including a pressurization channel of the suction cup for disengaging it.

FIG. 6 of sheet 2/3 shows a cross-section view of a prosthesis placed at the time of its introduction into the vertebral disk cavity, the vertebras having been slightly spread to facilitate its introduction.

FIGS. 6a and 6b show the details of two types of suction cups before they adhere to the wall.

FIG. 7 of sheet 2/3 shows a cross-section view of a prosthesis placed in the cavity of an intervertebral disk, after release of the spread of the vertebras.

FIGS. 7a and 7b show the details of two types of suction cups once that they adhered to the wall

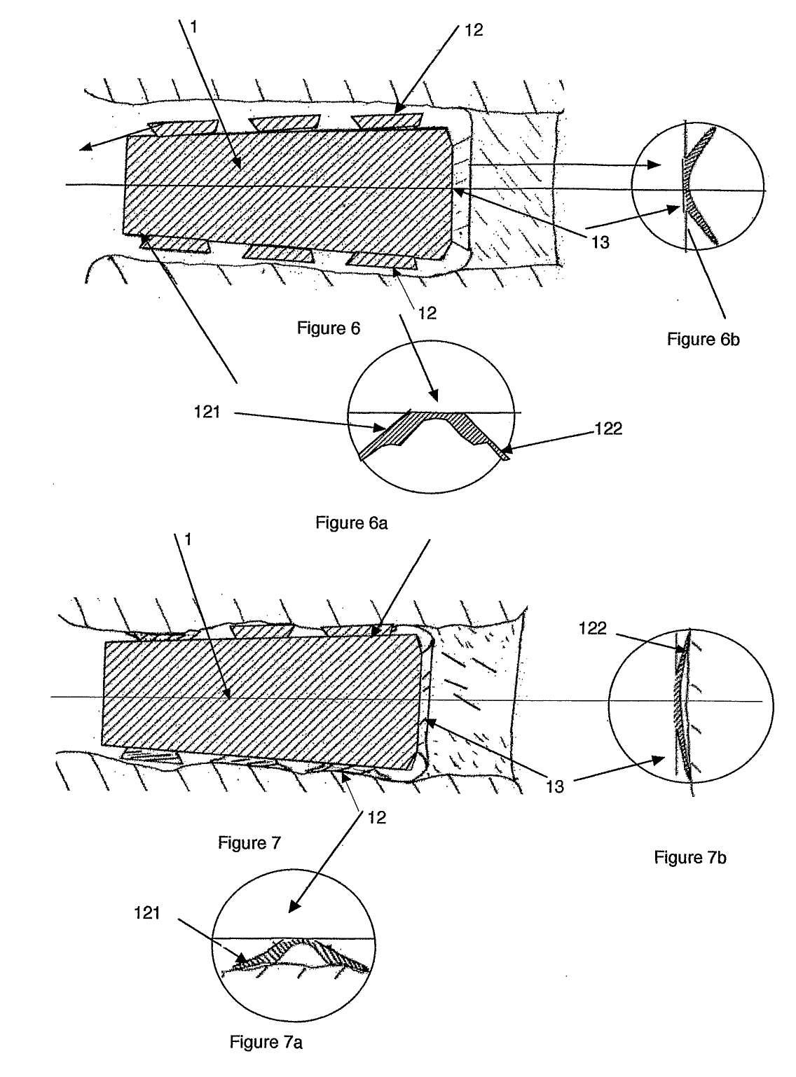

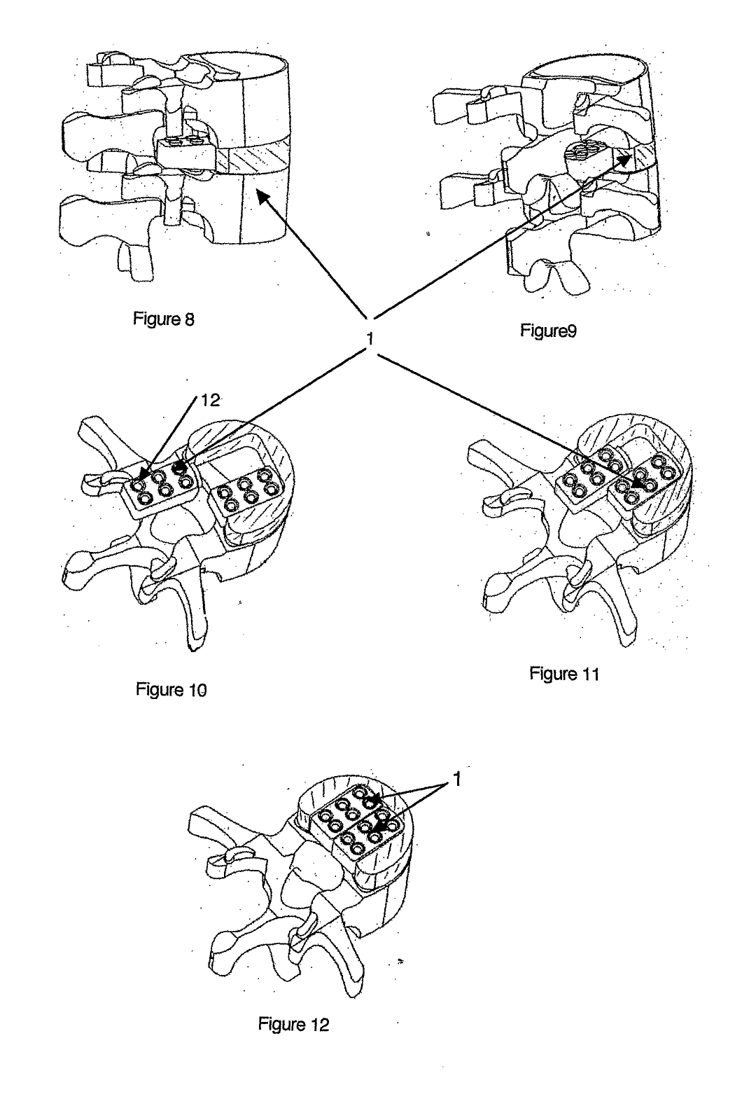

FIGS. 8 and 9 of sheet 3/3 show perspective views of a prosthesis at the time of its introduction into the vertebral body, here at two different angles.

FIGS. 10 and 11 of sheet 3/3 show two types of cross-section views in the area of two prosthesis, at the time of their introduction into the vertebral body, here at two different alignments of a prosthesis with respect to the other.

FIG. 12 of sheet 3/3 shows a perspective view of two installed prostheses, here in the cut plane of the inserted prostheses.

The adhesive and shock-absorbing prosthesis 1 includes two independent means placed side by side, perfectly integrated, and thus composed:

of a homogeneous block 11 made of a flexible and shock-absorbing material resistant to the compressive forces, polyurethane type material able to withstand the repeated mechanical stresses of the intervertebral disk

at least an adhesion means 12 or 13, the functionality of which is to have a suction cup effect enabling attainment of a flexible attachment of the prosthesis (1)

The homogeneous block 11 has a general polyhedron shape having at least six faces of which at least one face has suction cups 12 or the 13.

The two lateral faces 112 are preferably planes and mutually parallel.

The two inferior and superior faces 111a and b will be in contact with the adjacent vertebral bodies and subjected to compressive forces. Their two planes 111a and b form an angle Ω between them, which produces an effect of wedging into the corner which tends to on the one hand to oppose the expulsion and on the other hand to restore the lordosis of the patient.

These two inferior and superior faces 111a and b may or may not comprise means of adhesion, preferably suction cups 12, but possibly hollow surface means or teeth of the same type as the tread designs that one finds on vehicle tires, known means but whose application here is completely different. The prosthesis 1 is sandwiched. It has a corner shape that facilitates its wedging between the two faces of the vertebras.

The anterior face 113 of the homogeneous block 11 is put in contact with the elastic tissues of the annulus of the intervertebral disk. It will be able to benefit from the presence of at least one suction cup 13 that will adhere to the anterior wall of the intervertebral annulus by a pressure exerted by the surgeon at the time of its installation (FIGS. 8, 9, and 10).

The posterior face 114 of the homogeneous block 11 will receive the means of installation and of extraction of the adhesive and shock-absorbing prosthesis 1, which includes:

an opening 21 or a point of mechanical weakness in the material 22 is provided at the area of the face 113 situated at the end of a conduit 20 which start from the face 114 towards suction cup 13 to facilitate its disengagement, if one wishes to extract the prosthesis 1.

In a first implementation form (FIGS. 1 and 2) the adhesive means are preferably suction cups 12 positioned on each face 111a and b. These suction cups 12 have the characteristic of having small, thick resistant lips, the preferably conical shape of which will be particularly adapted to the vaulted and irregular surfaces of the vertebral plates. This conical shape also enables fitting in angles.

During the insertion phase of the prosthesis 1 (FIG. 8 to 12), it is appropriate to withdraw the dilapidated tissue from the implantation site that no longer adhere with the intervertebral disk, ligamentary, or bone cells, so that the suction cups can stick only on healthy tissues.

To facilitate the introduction of the prosthesis, it is necessary to slightly spread the plates of the vertebras (FIG. 6) with an appropriate instrument, in order to avoid the friction of the suction cups on their contact faces with the vertebras.

First, the lips of suction cups 12 and 13 are preferably molded in the material of the prosthesis at the same time as the homogeneous block 11.

Two types of suction cups are envisioned:

Suction cup 12 has a slightly conical shape, its adhesion surface being dimensioned to slightly deform under substantial mechanical loads induced by the vertical column.

The end 122 has a rather fine cross-section in order to be very easily squeezed and to ensure an effective joining with surrounding tissues even if the surface is irregular.

Suction cup 13 has a slightly conical shape. Its adhesion surface is preferably cylindrical. Its lip 123 is higher and more easily deformable than that of suction cup 12. Its cross-section has been designed in order to deform easily, pressing it on a more or less irregular elastic surface.

The choice of the distribution of suction cups 12 and 13 is a function of the cavity to be treated. These suction cups also have different adhesion characteristics described above and are selected according to the elasticity of tissues concerned, the pressures that they can sustain, as well as level of the pressure to be applied to obtain a good adhesion on the tissues.

To consider all the scenarios and to sure of the adhesion of at least one suction cup 12 or 13, various types of configurations are recommended, for example FIG. 2. These distributions of suction cups may or may not to be regular. The contact zones of the prosthesis with its implantation site, faces 113, and 111a it and b will be favored.

This distribution is always chosen in order that at least one suction cup 12 or 13 adheres, whatever the shape of the cavity to be filled. It is possible to add devices facilitating the extraction of the prosthesis 1. This extraction device for the prosthesis 1 can to be constituted by for example: of at least one small channel 20 (FIG. 5) designed to receive a light overpressure in relation to the atmospheric pressure that will allow disengaging suction cup(s) 13 adhering to the annulus at the bottom of the cavity.

This channel 20 that crosses the prosthesis is provided with a small stopper 21 that seals conduit 20 avoiding an unforeseen disengagement of the suction cup 13 during its operation. One can also provide a mechanical fusible plug 22, at the end of channel 20 at the entrance of the suction cup, that will be able to be perforated by an instrument.

This adhesive prosthesis 1 requires the use of an insertion device: it is a tool causing a very slight spread of the plates of the vertebras intended to facilitate the insertion of the adhesive prosthesis 1, by avoiding a forceful introduction that could damage the suction cups

As soon as this spreading device is retracted, the suction cups 12 13 situated on the faces 111a and b are automatically placed under pressure, under the effect of the restoring elastic forces exerted by the annulus and the ligaments connecting the vertebras. The lips 122 deform are deformed and immediately adhere to the wall.

The prosthesis 1 is completely stabilized. It can operate with the aid of its mechanical behavior and shock absorbing characteristics according to its material of constitution. This involves, as opposed to the other devices, a very good stability, which improves as the exterior mechanical stresses are elevated; in fact, the more the lips of suction cup are squeezed, the stronger the adhesion.

Suction cups 12 and 13 and the other means previously described, which could have as the means 12 and the 13 the adhering functionality by suction cup effect, are directly molded in the same material a the prosthesis 1 used to realize such integrated prostheses, and which is perfectly appropriate for its mechanical and shock-absorbing properties.

1. Adhesive, shock-absorbing intervertebral disk prosthesis including an adhesion means characterized in that it is made of two independent homogenous blocks realized in a single polyurethane type material resistant to the dynamic and repeated mechanical stresses of the intervertebral disk.

2. Adhesive, shock-absorbing intervertebral disk prosthesis according to claim 1 including adhesion means constituted by suction cups distributed on two faces of the blocks characterized in that each of the blocks has a general polyhedron shape having six faces of which:
two lateral faces are planes and mutually parallel
two inferior and superior faces are placed in contact with the adjacent vertebral bodies and form an angle between themselves, which produces an effect of wedging that opposes the expulsion of the blocks that constitute the prosthesis.

3. Adhesive, shock-absorbing intervertebral disk prosthesis according to claim 2 characterized in that includes an opening or a point of mechanical weakness in the material provided at the area of the face situated at the end of a conduit which extends from the face towards suction cup to facilitate its disengagement.

4. Adhesive, shock-absorbing intervertebral disk prosthesis according to claim 1 characterized in that the adhesion means include suction cups positioned on each face, these suction cups having the characteristic of having small, thick resistant lips, having preferably conical shape for adapting more particularly to the vaulted and irregular surfaces of the vertebral plates, this conical shape also enabling fitting in angles.

5. Adhesive, shock-absorbing intervertebral disk prosthesis according to claim 1 characterized in that the suction cup has a slightly conical shape, its adhesion surface being dimensioned to slightly deform under substantial mechanical loads, the aforementioned suction cup having an end having a rather fine cross-section enabling an easily squeezing and to ensure an effective joining with surrounding tissues even if the surface is irregular.

6. Adhesive, shock-absorbing intervertebral disk prosthesis according to claim 1 characterized in that the suction cup having a slightly conical shape, has a cylindrical adhesion surface, its lip being higher and more easily deformable than that of suction cup; Its cross-section being able to deform easily, pressing it on a more or less irregular elastic surface.

7. Adhesive, shock-absorbing intervertebral disk prosthesis according to claim 1 characterized in that the suction cup has a slightly conical shape, its adhesion surface being dimensioned to slightly deform under substantial mechanical loads, the aforementioned suction cup having an end having a rather fine cross-section in order to be very easily squeezed and to ensure an effective joining with surrounding tissues even if the surface is irregular.

8. Adhesive, shock-absorbing intervertebral disk prosthesis according to claim 1 characterized in that the suction cup, having a slightly conical shape, has a cylindrical adhesion surface, its lip being higher and more easily deformable than that of suction cup; its cross-section being able to deform easily by supporting it on a more or less irregular elastic surface.
